(19) United States
(12) Patent Application Publication
(10) Pub. No.: US 2022/0379310 A1
(43) Pub. Date: Dec. 1, 2022

(54) FLUIDIC PROCESSING WORKSTATION

(71) Applicant: FloDesign Sonics, Inc., West Springfield, MA (US)

(72) Inventors: Brian Dutra, Granby, CT (US); Christopher Jurkiewicz, Ellington, CT (US); Brian McCarthy, East Longmeadow, MA (US); Richard Grant, St. Kilda West (AU); Jeffrey King, Coventry, CT (US); Bart Lipkens, Bloomfield, CT (US); Yasser Ali, Westborough, MA (US); Stefano Berti Perez, Boston, MA (US)

(21) Appl. No.: 17/804,211

(22) Filed: May 26, 2022

Related U.S. Application Data
(60) Provisional application No. 63/194,166, filed on May 27, 2021.

(51) Int. Cl. B01L 3/00; C12N 5/078

(52) U.S. Cl. CPC B01L 3/502761; C12N 5/0634; B01L 2400/0436; B01L 2400/0633; B01L 2200/025; B01L 2300/0663; B01L 2400/0481; B01L 2200/0652

(57) ABSTRACT

A workstation for processing particles entrained in a fluid includes a consumable portion and a reusable portion. The consumable portion is mounted to the reusable portion to form a fluid chamber in which an acoustic wave can be generated. The consumable portion implements a closed, isolated fluid environment that is managed using components of the reusable portion, such as valves, sensors and pumps. The workstation can be operated to retain particles from the fluid via the acoustic wave and provide a new fluid media to the retained particles. Following processing, the consumable portion can be removed and discarded.

### CROSS-REFERENCE TO RELATED APPLICATIONS

The present application claims the benefit of priority of U.S. Provisional Patent Application No. 63/194,166, filing date May 27, 2021, the entire contents of which is hereby incorporated herein by reference in its entirety.

### BACKGROUND

Devices and methods for processing cells and cellular material have been implemented in life science related industries. Production examples include monoclonal antibodies and proteins for therapeutic compounds, genetic material, viral material and virus vectors and cells and cellular material for cell therapy applications, to name a few. The class of above devices and methods applicable to life science related industries are referred to collectively herein as cell processing.

Separation of biomaterial is used in cell processing in a variety of contexts. For example, separation techniques for separating proteins from other biomaterials are used in a number of analytical processes. Separation of desired cells from a general cell population is used in cell processing, as is separation of cells or cell cultures from a host fluid.

Concentrating therapeutic cells and transferring them from one solution into another (sometimes referred to as washing or media exchange) are two processes implemented at multiple stages of production and use of such cells. The washing and separation of materials in cellular processing is an important part of the overall efficacy of the cell therapy of choice. In particular, therapeutic cells may originally be suspended in a growth serum or in preservative materials like dimethyl sulfoxide (DMSO). Separating the cells from these fluids so the cells can be further processed is important in the overall therapeutic process of using such cellular materials. In one example, the cells are recovered from a bioreactor, concentrated, and transferred from culture media into an electroporation buffer prior to transduction, such as may occur in the manufacture of CAR-T cells. After expansion of cells in a later manufacturing step, the cells are concentrated and transferred into an appropriate solution, the makeup of which depends on the desired application for the cells.

Therapeutic cells are stored in specialized media to prolong the viability of these cells either through refrigeration and/or freezing processes. Such specialized media may be sought to be removed when the therapeutic cells are introduced into a patient. It may thus be helpful to both wash and concentrate the therapeutic cells in a buffer or wash media that is biocompatible with both the therapeutic cells and with the patient. These washing and concentration processes conventionally involve the use of centrifugation and/or membrane filtration. The washing step may be repeated a number of times. For example, the specialized media (which can be pyrogenic or otherwise harmful) may be fully removed with multiple wash steps, and the cells may be suspended in a new buffer or wash solution. During this washing process, many of the cells are degraded or destroyed through the centrifugation and/or membrane filtration processes. Moreover, the filtration process can be rather inefficient and may entail a non-sterile intrusion into the environment for batch processing, whereby the cell culture is exposed to possible pathogens or outside cellular influences that would be harmful to the target cell culture. In addition, the filtration processes may generate biological waste through the use of multiple filters which may incur additional steps or cost for proper disposal. The above noted factors may increase cost and processing times for the preparation of therapeutic cells for introduction to the patient.

### BRIEF DESCRIPTION

A fluidic processing workstation is configured to process particles, which may be biological materials, such as apheresis material, monoclonal antibodies, proteins, genetic material, viruses, viral vectors, cells or cellular material as examples. The workstation may implement one or more particle processing operations, including cell processing operations, such as, for example, cell separation from a host fluid, cell selection from a general cell population, cell retention, concentration or washing or media exchange. The workstation includes a reusable portion and a single use or consumable portion for implementing the particle processing operations. The workstation is provided with a user interface for controlling processes and operations implemented with the workstation.

The reusable portion of the workstation may include a fluid handling unit (FHU) and a system control unit (SCU). The FHU is provided with equipment for controlling and/or measuring fluid parameters and includes an ultrasonic transducer for generating an acoustic wave. A cooling loop for cooling the ultrasonic transducer may optionally be provided in the FHU. The FHU equipment may include, for example, one or more pumps, valves, sensors and/or interfaces for fluid and electronics management. The SCU is provided with equipment for controlling and managing the FHU and SCU components and operation. For example, the SCU includes one or more acoustic driver modules (ADMs) that each provide a drive signal operating an ultrasonic transducer, such as an ultrasonic transducer in the FHU. The SCU also provides resources for implementing a graphic user interface (GUI). The GUI is mounted to the SCU, and provides an interface for users to set process parameters and for presenting information about processes.

The consumable portion of the workstation includes a fluid processing element (FPE) that can be connected to the FHU. The FPE includes a self-contained fluid environment implemented with fluid chambers and tubing that can interface or engage with corresponding components on the FHU. For example, the FPE includes tubing that can be coupled to a pump to permit fluid to be pumped through the FPE. Some tubing in the FPE can be coupled to a valve, such as a pinch valve, on the FHU to permit or prevent fluid flow in the coupled tubing. The FPE includes a reflector that cooperates with an ultrasonic transducer on the FHU to contribute to forming an acoustic field in a fluid chamber of the FPE. The FPE may optionally be mounted to the FHU. The FPE may include mounting and/or alignment structures that cooperate with corresponding structures on the FHU to permit mounting of the FPE in a desired registration with the FHU. For example, the mounting and/or alignment structures on the FPE contribute to registering a section of tubing with a valve on the FHU, and/or position the reflector in a desired alignment with the ultrasonic transducer on the FHU. The FPE may be configured to implement one or more processing operations, such as, for example, cell selection, cell concentration, cell retention and/or cell washing.

In some example implementations, the workstation employs equipment and techniques for washing particles, which may be cells. In some example methods, an initial mixture of a first media and the particles is fed to a fluid chamber of the FPE, which is operatively coupled to the FHU. The first media may contain preservatives such as dimethyl sulfoxide (DMSO) which are undesirable for future applications/uses of the particles. The FHU includes at least one ultrasonic transducer that includes a piezoelectric element and is configured to be driven to generate an acoustic wave in the fluid chamber of the FPE. The piezoelectric element may be driven to produce a uniform displacement, causing acoustic waves to be generated in a single direction in the fluid chamber. The piezoelectric element may be driven to produce a non-uniform displacement, causing acoustic waves to be generated in multiple directions in the fluid chamber. The acoustic waves produce an acoustic field in the fluid chamber that produces acoustic radiation forces on the particles, causing the particles to be trapped, retained and/or clustered in the acoustic field. A second media is flowed into the fluid chamber to wash out the first media while the particles are retained in the acoustic field. The particles may thus experience a media exchange, where the first media is exchanged for the second media.

In some examples, the volume of the second media used to perform the wash process may be equivalent to a volume of the fluid chamber. In some examples, the volume of the second media used to perform the wash process may be multiples of or portions of the volume of the fluid chamber. The second media can be a biocompatible wash or a buffer solution.

The particles may be cells. The cells may be Chinese hamster ovary (CHO) cells, NS0 hybridoma cells, baby hamster kidney (BHK) cells, human cells, regulatory T-cells, Jurkat T-cells, CAR-T cells, B cells, or NK cells, peripheral blood mononuclear cells (PBMCs), algae, plant cells, bacteria, or viruses. The cells may be attached to structures such as, for example, beads, droplets, microcarriers, bubbles, or any other kind of structure that can support a cell attachment. The structures may be functionalized with material that has an affinity for certain cells, cell types or features provided to cell surfaces or membranes.

In some example implementations, the ultrasonic transducer includes an array formed from a plurality of piezoelectric elements. Each piezoelectric element can be physically separated from surrounding piezoelectric elements by a potting material. The piezoelectric array can be present on a single crystal, with one or more channels separating the piezoelectric elements from each other. Each piezoelectric element can be individually connected to its own pair of electrodes. The piezoelectric elements can be operated in phase with each other, or operated out of phase with each other. The FHU may further comprise a cooling unit for cooling the ultrasonic transducer.

In various embodiments, the initial mixture may have a density of about 0.5 million particles/mL to about 5 million particles/mL. Depending on the initial volume, the concentrated volume can be about 5 to about 200, about 10 to about 100, or about 25 to about 50 times less than a volume of the initial mixture. The concentrated volume may have a particle density of about 5 to about 250, about 10 to about 100, or about 25 to about 50 times greater than a particle density of the initial mixture. In some examples, the final volume can be about 5 ml, or in a range of from about 1 ml to about 100 ml, 2 ml to about 50 ml, or about 5 ml to about 25 ml. In the case of an apheresis product, red blood cells (RBC), white blood cells (WBC) and platelet concentration can be significantly higher than other particles. In some examples, the incoming volumes can be from about 100 ml to about 5 liters, about 200 ml to about 2 liters, or about 250 ml to about 1 liter. Total initial cell count can be from several hundred million total cells to tens of billions cells. The washout, selection or retention efficiency of the workstation can be better than 95%

Various example implementations include methods of recovering greater than 90% of cells from a cell culture. An initial mixture of a first media and the cell culture is fed to the fluid chamber of the FPE. The ultrasonic transducer of the FHU is driven to generate an acoustic wave in the fluid chamber, thereby forming an acoustic field in the fluid of the fluid chamber. The acoustic field imposes forces to concentrate the cells at locales within the acoustic field. These locales may have reduced levels of pressure and/or acoustic radiation forces compared with other locales in the acoustic field. The initial mixture has an initial cell density of about 0.5 million cells/mL to about 5 million cells/mL, and the concentrated cell culture has a cell density at least 25 times greater than the initial cell density.

In some embodiments, the concentrated cell culture has a cell density of 25 to about 50 times greater than the initial cell density. In other embodiments, a volume of the concentrated cell culture is 25 to about 50 times less than a volume of the initial mixture. The concentrated cell culture can be obtained in about 35 minutes or less.

The FPE may include a fluid chamber with a fluid inlet, a first outlet, and a second outlet. In some examples, a port is provided that is used as an inlet and an outlet, such as when wash fluid is introduced into the fluid chamber and concentrated cells or waste is removed. The fluid chamber may be bordered by the reflector. The reflector may be implemented as a faceted reflector that serves to disperse or scatter an incoming acoustic wave.

The FPE may be configured to produce a concentrated volume of about 1 ml to about 100 ml, about 5 ml to about 75 ml, or about 25 ml to about 50 ml. In some examples the concentrated volume is 5 ml. The FPE may have a cell capacity of about 4 billion to about 40 billion cells. Various lines can connect the FPE to containers that provide or receive various materials to/from the fluid chamber.

The SCU may be configured to produce and RF drive signal that is provided to the ultrasonic transducer in the FHU via an RF cable, such as, for example, a coaxial cable. The ultrasonic transducer may be driven at a frequency in an inclusive range of from about 0.5 MHz to about 4 MHz, or at a frequency in a range of below or above about 1.5 MHz.

In particular constructions, the at least one inlet is part of a dump diffuser. The at least one inlet may be located at a height between 5% and 75% of a height of the acoustic chamber. The at least one inlet may be in the shape of holes or slots that provide an initial flow direction parallel to the acoustic wave generated by the ultrasonic transducer. The device may include a shallow wall below the at least one inlet and leading to the at least one outlet, wherein the shallow wall has an angle of 60° or less relative to a horizontal plane.

In some examples, two inlets are provided on opposite sides of the fluid chamber. One or more inlets are below the acoustic field. In some examples, a collector region for collecting concentrated material is provided below an inlet region, where the inlet(s) are provided. The inlet region is below an acoustic region where the acoustic field is established. An outlet region is provided above the acoustic region and is provided with an outlet. The outlet may be used to collect concentrated material or waste. The collector region is provided with a port that may be operated as an inlet or outlet. The port may be used to collect concentrated material or waste.

The fluid chamber of the FPE may be reflectionally symmetrical through a vertical plane. The at least one inlet may include a plurality of inlets located at different points about the fluid chamber, such that the inflow of the mixture into the fluid chamber is locally consistent and symmetrical. The fluid flowed into the fluid chamber may consist of a mixture of a host fluid and a second fluid or particulate. The fluid chamber and fluid passageways of the FPE are configured to permit the mixture to flow into and through the FPE at a rate of at least 4.65 ml/minute per cm² or in a range of from about 1 ml/min to about 1 liter/min, about 2 ml/min to about 500 ml/min, or about 5 ml/min to about 250 ml/min.

The particulate may be Chinese hamster ovary (CHO) cells, NS0 hybridoma cells, baby hamster kidney (BHK) cells, or human cells, T cells, B cells, NK cells, algae, bacteria, viruses, apheresis material, iPSCs, hPSCs, or other cells grown as aggregates, or microcarriers.

Also disclosed in various embodiments herein are acoustophoresis devices comprising: a housing having a sidewall that defines an acoustic chamber; and at least one ultrasonic transducer located on the sidewall of the acoustic chamber and at least one reflector located on the sidewall of the housing opposite the at least one ultrasonic transducer, the transducer including a piezoelectric material driven by a voltage signal to create a multi-dimensional acoustic standing wave in the acoustic chamber, resulting in a set of trapping lines in the acoustic chamber, the transducer being oriented to minimize cross-sectional area for straight vertical channels between the trapping lines. This can be done as described herein.

Also disclosed are methods of separating a host fluid from a second fluid or particulate, the method comprising: flowing a mixture of the host fluid and the second fluid or particulate through an acoustophoresis device in a uniform fashion, the device comprising: a housing having a sidewall that defines an acoustic chamber; at least one ultrasonic transducer located on the sidewall of the acoustic chamber and at least one reflector located on the sidewall of the housing opposite the at least one ultrasonic transducer, the transducer including a piezoelectric material driven by a voltage signal to create a multi-dimensional acoustic standing wave in the acoustic chamber, resulting in a set of trapping lines or trapping zones or trapping locales in the acoustic chamber, the transducer being oriented to minimize cross-sectional area for straight vertical channels between the trapping lines; and capturing smaller particles of the second fluid or particulate in the trapping lines to cluster and continuously gravity separate the second fluid or particulate from the host fluid.

These and other non-limiting characteristics are more particularly described below.

### BRIEF DESCRIPTION OF THE DRAWINGS

The following is a brief description of the drawings, which illustrate example implementations presented herein.

FIG. 1 is an isometric view of the workstation equipped with a consumable cartridge and fluid source bags and receptacles.

FIG. 2 is an isometric front view of the SCU.

FIG. 3 is an isometric rear view of the SCU.

FIG. 4 is an isometric view of the SCU with the cover removed and showing a portion of internal components.

FIG. 5 is an isometric front view of the FHU without a consumable cartridge installed.

FIG. 6 is an isometric rear view of the FHU.

FIG. 7 is an isometric view of the FHU with the cover removed and showing internal components.

FIG. 8 is an isometric view of the FHU of FIG. 7 with a portion of internal components being removed.

FIG. 9 is a front elevation view of the FPE.

FIG. 10 is a front elevation view of the FHU without a consumable cartridge installed and showing FPE interface components.

FIG. 11 is a rear elevation view of the FPE.

FIG. 12 is a rear elevation view of the FPE with the rear cover removed to show the internal configuration of the FPE.

FIG. 13 is a front elevation view of the FHU with a portion of the FPE superimposed on the front interface panel and a sectional view of the portion of the FPE.

FIG. 14 is a sectional diagram of an example implementation of the fluid chamber of the FPE with a faceted reflector and with a planar reflector.

FIG. 15 is a sectional diagram of an example implementation of the fluid chamber of the FPE in low cell density operation and in high cell density operation.

FIG. 16 is a diagram of an example implementation of FHU components and a fluid path through the FPE for feed material.

FIG. 17 is a diagram of the implementation of FIG. 16 showing a fluid path through the FPE for wash material.

FIG. 18 is a diagram of the implementation of FIG. 16 showing a fluid path for draining material from the FPE.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, i.e. the surface of the earth. The terms “upwards” and “downwards” are also relative to an absolute reference; upwards is always against the gravity of the earth.

The term “parallel” should be construed in its lay sense of two surfaces that maintain a generally constant distance between them, and not in the strict mathematical sense that such surfaces will never intersect when extended to infinity.

The present application refers to “the same order of magnitude.” Two numbers are of the same order of magnitude if the quotient of the larger number divided by the smaller number is a value less than 10.

The various implementations and techniques discussed herein relate to fluid processing devices and operations that employ acoustic fields to manipulate particles entrained in a host fluid. As used herein, particles refer to discrete units of material that can be solid, liquid or gaseous, and that can be relatively rigid, flexible or compressible, that may include subcomponents or that may be configured as a combination of materials with one or more of the preceding characteristics. The acoustic fields include locales of relatively lower pressure and/or acoustic radiation forces that tend to collect the particles. In some example implementations, the acoustic field produces tightly packed clusters of particulates which continuously drop out or rise out of a flowing fluid mixture due to gravity and/or buoyancy forces.

Referring to FIG. 1, the fluidic processing workstation includes a system control unit (SCU) located on top of a fluid handling unit (FHU). The SCU and FHU represent reusable components for implementing a fluid and particle processing system. Mounted on a front panel of the FHU is a fluid processing element (FPE) that is single use, disposable or consumable. The SCU includes a graphic user interface (GUI), which can be implemented as a touchscreen display. The angle of the display is adjustable for convenience of individual users. The SCU is connected to the FHU with various electrical connectors. The FHU includes a front panel configured with components that engage or interface with complementary components of the FPE. The FPE provides a tubing configuration to permit interaction with the FHU front panel interface. The tubing configuration in the FPE contributes to reducing or eliminating tubing path set up errors to provide a simplified approach to establishing fluid management and control of the fluid processing implemented in the FPE. The set up for a fluid processing operation includes mounting the FPE on the front panel interface of the FHU, which provides engagement of the desired valves and pumps with the appropriate tubing locations to control and manage fluid processing in the FPE. The approach implemented with the consumable cartridge mounted to the reusable FHU interface permits rapid and accurate set up for a fluid processing that can be configured for a number of different operations through the control functions provided by the SCU and FHU. The FHU may optionally include a cooling loop coupled to the ultrasonic transducer to remove heat generated during operation.

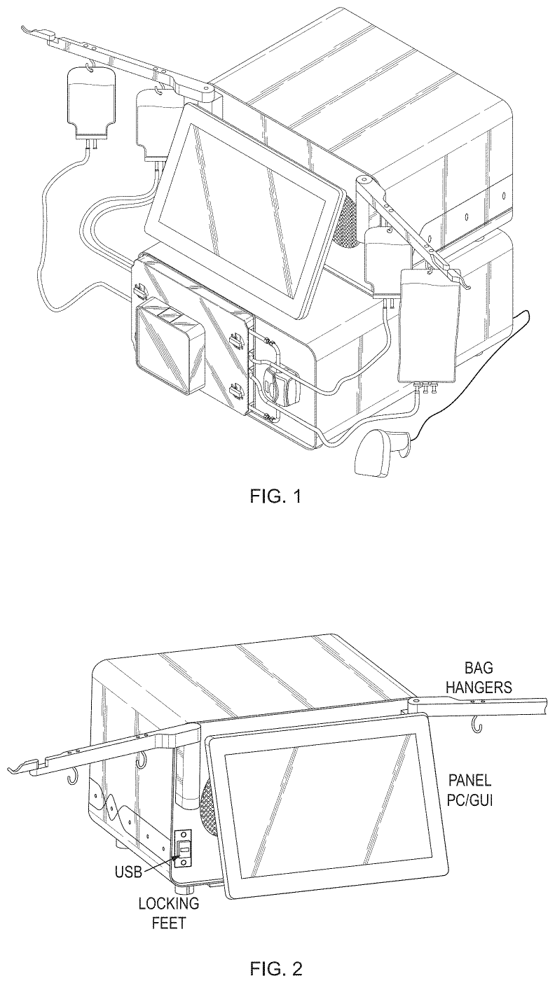

FIG. 2 illustrates the SCU viewed from the front and showing the display, which can be implemented as a touchscreen display. The base of the SCU can include locking feet for mounting the SCU on top of the FHU in a stable or nested configuration. A USB port can be provided on the SCU to permit data exchange with external devices, such as may be desirable for software or firmware upgrades or exporting stored data. The SCU is optionally equipped with rotating arms that can be extended to act as hangers for bags that may be provided as fluid sources or receptacles.

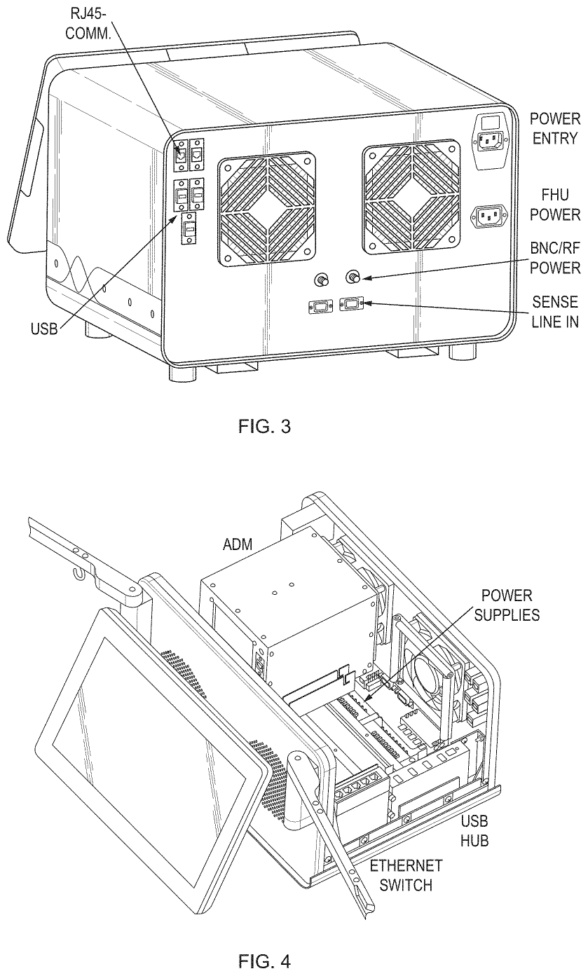

FIG. 3 illustrates a rear view of the SCU showing various connection points and cooling fans. The SCU includes a power receptacle that can be connected to standard building power sockets available in North America, Europe and other locations. Power provided to the SCU is used to supply power to the FHU through a power output receptacle. Accordingly, the fluid processing workstation can be operated with power provided at a single input point. RJ-45 communication connections are provided on the SCU to permit communication with multiple FHUs. BNC connectors for RF power cables used to connect to and drive ultrasonic transducers in multiple FHUs are provided. The RF power cables can be implemented as coaxial cables. Multiple USB connectors are provided for peripheral interfacing, such as may be used with a barcode scanner, keyboard or mouse. Multiple connection interfaces for feedback and fault monitoring information provided from multiple FHUs are arranged on the rear of the SCU. The SCU includes several fans to remove excess internally generated heat.

FIG. 4 illustrates an internal configuration of the SCU with some components removed for visibility. The SCU includes multiple acoustic driver modules (ADMs), each of which is configured to generate an RF signal that is supplied to the ultrasonic transducer of an FHU. Accordingly, each SCU can provide an RF power output to at least two FHUs to drive the corresponding ultrasonic transducers. The SCU includes power supplies for driving the ADMs, for example, a 36 V DC power supply being provided for each ADM. The SCU may include a power supply for powering other components in the SCU, which may, for example, be implemented as a 12 V DC power supply. The SCU may include an ethernet switch that permits communication between an individual ADM and a connected FHU. A USB hub may be provided to the SCU for communication in connection with peripherals, such as a barcode scanner, keyboard or mouse device.

Each ADM is configured to generate an RF power signal that drives the ultrasonic transducer in an FHU. The ADM uses a DC input that is applied to a DC-DC converter to control a power level of the RF output. The output of the DC-DC converter is applied to an RF power inverter that generates an RF signal that is used to drive an ultrasonic transducer in an FHU. The ADM includes a control board that is configured to receive feedback from the ultrasonic transducer and control the DC-DC converter and RF power inverter to produce a desired RF power signal output. An FPGA and other firmware may be implemented on the control board, which permits control program updates to be implemented as desired. A system board is included in the ADM to simplify interfacing with other electronic boards, such as RF or control boards. The system board may provide interconnection with the RJ-45 communication interface, the fault monitoring feedback signal or other system functions.

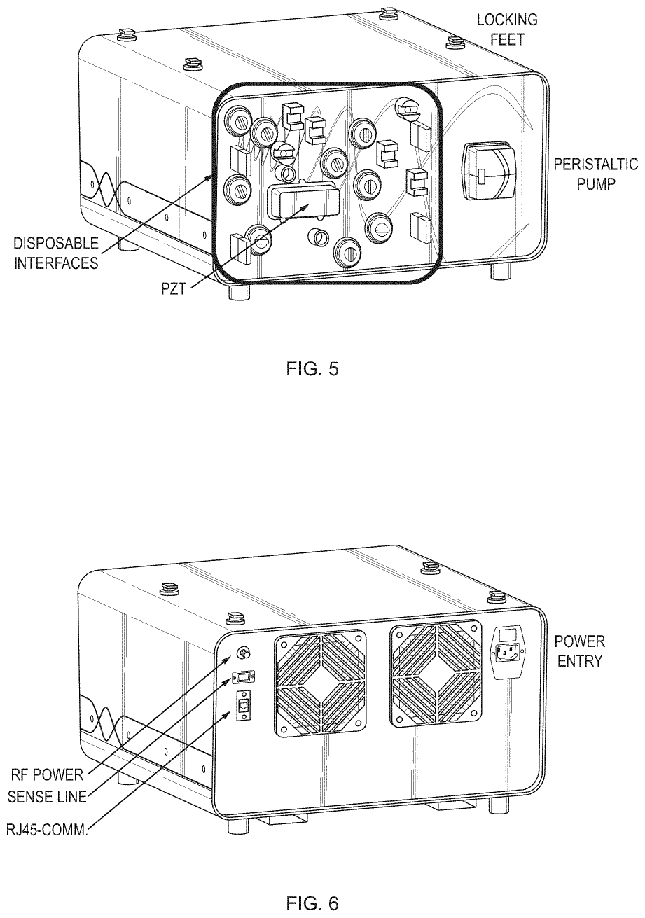

FIG. 5 shows an FHU with a front panel interface that includes an ultrasonic transducer and peristaltic pump. The front panel interface is configured with components that mate and cooperate with corresponding elements of an FPE mounted to the front panel interface. The front panel interface includes an ultrasonic transducer (PZT), nine solenoid valves, four bubble sensors, two infrared temperature sensors, a cooling loop inlet and outlet and four locking latches that retain the FPE on the front panel interface. The arrangement of the various components on the front panel interface permits their engagement with corresponding components of the FPE to permit control and management of fluid being processed through the FPE. Accordingly, the valve, sensor and cooling loop components interact with tubing accessible via openings on the back of the FPE. The ultrasonic transducer interacts with the fluid chamber and reflector in the FPE. The FHU includes locking feet on a top portion of the case to receive cooperative locking feet of the SCU to permit the SCU to be stacked or nested on top of the FHU in a stable configuration.

FIG. 6 illustrates a backside of the FHU including electrical connection interfaces for electrical connections with the SCU. Power for the FHU is supplied from the SCU, which permits a single point of power supply to the workstation. Electrical connection interfaces are provided for RF power signal input, which is provided to the ultrasonic transducer, a sensing connection for providing feedback to the SCU from the ultrasonic transducer, and an RJ-45 communication interface to provide communication between the FHU and SCU. The FHU includes multiple rear exhaust fans for removing internally generated heat.

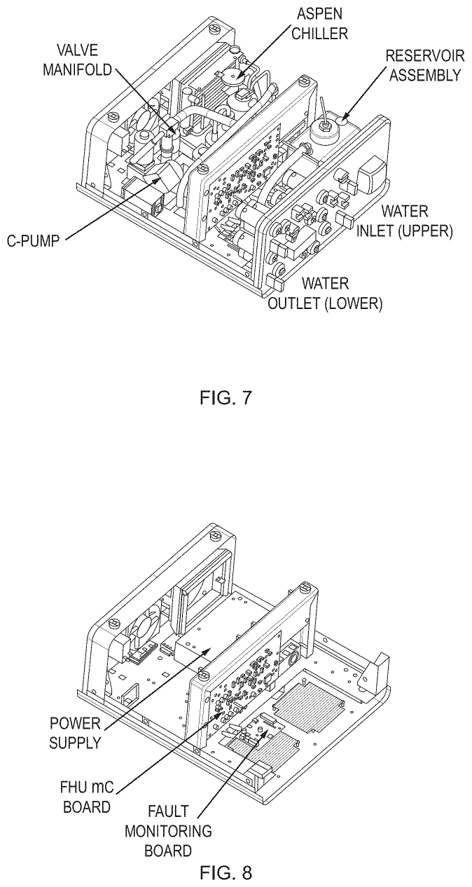

FIG. 7 shows an internal configuration of the FHU, with components for chilling cooling fluid for the ultrasonic transducer, as well as pumps and manifolds for providing the cooling fluid to the ultrasonic transducer and returning heated fluid. The FHU further includes a reservoir that can store cooling fluid. The cooling fluid is provided to an outlet on the front panel interface that is coupled to a fluid path in the FPE that passes the cooling fluid across the face of the ultrasonic transducer to draw away thermal energy and cool the ultrasonic transducer. The heated fluid is returned through the FPE to an inlet on the front panel interface of the FHU, where it is directed to the chiller to be cooled. The cooling configuration for removing thermal energy from the ultrasonic transducer can extract a significant amount of heat, thereby permitting a wide range of operating modes for the ultrasonic transducer.

FIG. 8 illustrates internal components of the FHU with the cooling fluid components removed. The components shown in FIG. 8 include an FHU power supply, for example, a 24 V DC power supply, a fault monitoring board for monitoring operation of the ultrasonic transducer and enunciating faults, and a microcontroller board for controlling operation of the FHU.

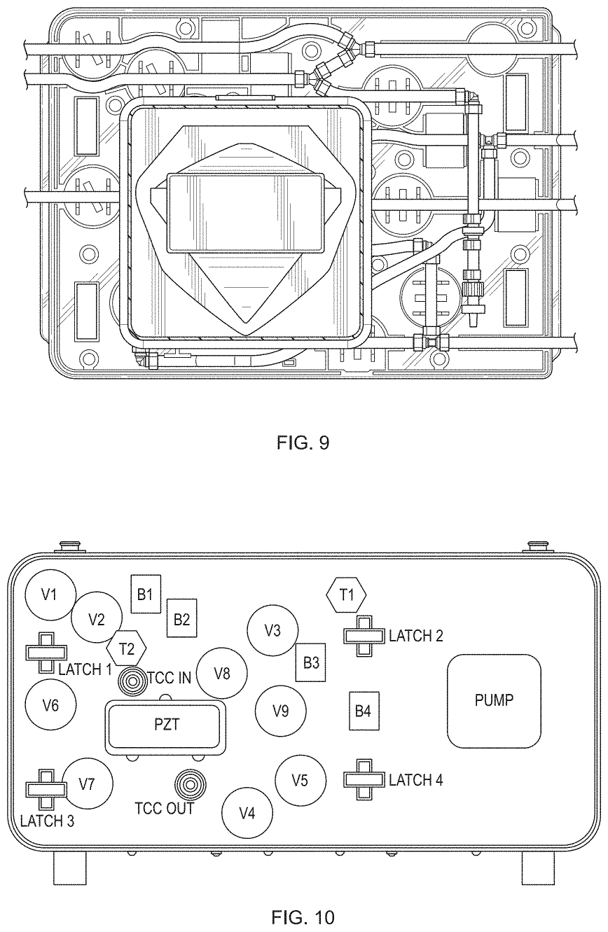

FIG. 9 is a front elevation view of the FPE showing the fluid chamber and reflector within a square module and tubing pathways and openings in the back of the FPE that coordinate with components on the front interface panel of the FHU. The FPE is implemented, for example, with transparent material to permit the operator to ensure proper alignment of the FPE tubing and openings with the components on the front interface panel of the FHU. In addition, the transparent material used to construct the FPE permits observation of the fluid chamber housed in the square module seen on the front of the FPE. The FPE pictured in FIG. 9 is used to implement a concentrate-wash operation for cell processing, described in greater detail below. The FPE is a single use, disposable or consumable component, which permits fluid management and processing in a closed, sterile environment, where components do not need to otherwise be sanitized or sterilized before reuse. In addition, because the FPE is a replaceable component, different types of configurations of the FPE can be implemented to realize different applications and processes. For example, a configuration for an FPE can be implemented that performs a particle separation, selection, retention and/or mixing operation, using the same front panel interface of the FHU. This approach and configuration of the fluid processing workstation provides significant flexibility for implementing acoustic based processes, while maintaining isolation of the fluid processed through the FPE. The FPE may include an inlet and outlet for a cooling loop to pass cooling fluid provided by the FHU over the face of the ultrasonic transducer for the removal of thermal energy. The FPE may be implemented without a cooling loop in applications where the ultrasonic transducer does not generate significant heat, in which case the FPE may provide seals to block fluid from the FHU cooling loop inlet or outlet.

FIG. 10 illustrates the front panel interface of the FHU. The illustrated larger circular components represent solenoid valves, the illustrated rectangular components represent bubble sensors, the illustrated hexagonal components represent IR temperature sensors, the illustrated smaller circular components represent a cooling loop inlet and outlet. The illustrated cross-shaped components are latches for securing the FPE to the FHU front panel interface. The latches operate in conjunction with through openings of the FPE, and include rotating sections that can be rotated to engage the front face of the FPE installed on the front panel interface to urge the FPE into contact and alignment with the front panel interface components. Also illustrated in FIG. 10 is the ultrasonic transducer (PZT) and peristaltic pump.

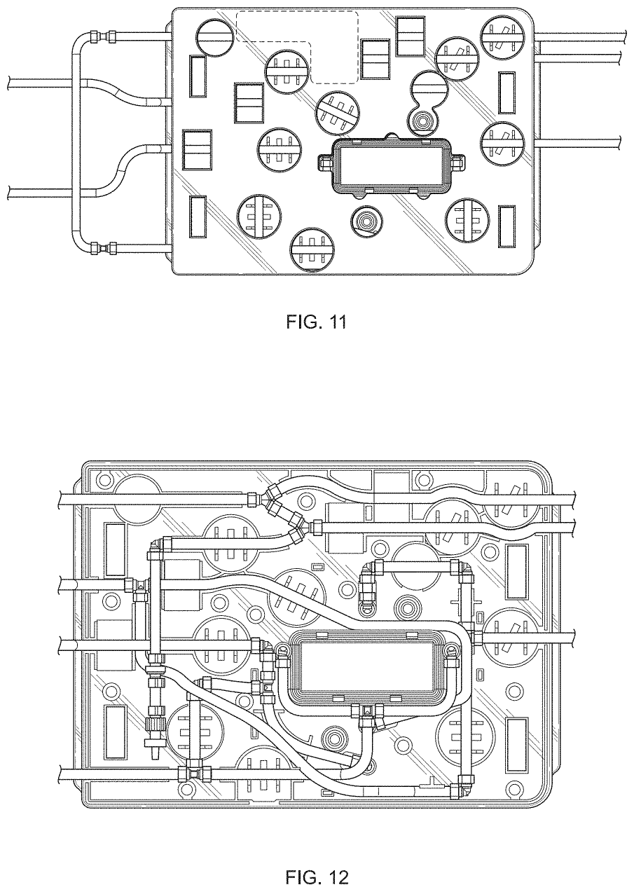

FIG. 11 is a rear elevation view of the FPE showing openings that permit access to the tubing within the FPE. Also shown is the inlet and outlet for the cooling fluid path that is configured to pass across the face of the transducer to remove thermal energy. Through openings for receiving the front panel interface latches are also illustrated as narrow vertical rectangles. A rectangular interface for receiving the ultrasonic transducer is also illustrated. The opposite side of the rectangular interface includes a reflector for reflecting an acoustic wave to contribute to generating an acoustic field within the fluid chamber of the FPE.

FIG. 12 is a rear elevation view of the FPE with the rear cover removed to show the internal configuration of the tubing. The tubing is arranged in a particular pattern to be able to cooperate with corresponding components on the front panel interface and to realize the fluid processing operation for which the FPE is designed. The tubing pattern illustrated in FIG. 12 is configured to permit implementation of a concentrate-wash operation, described in greater detail below.

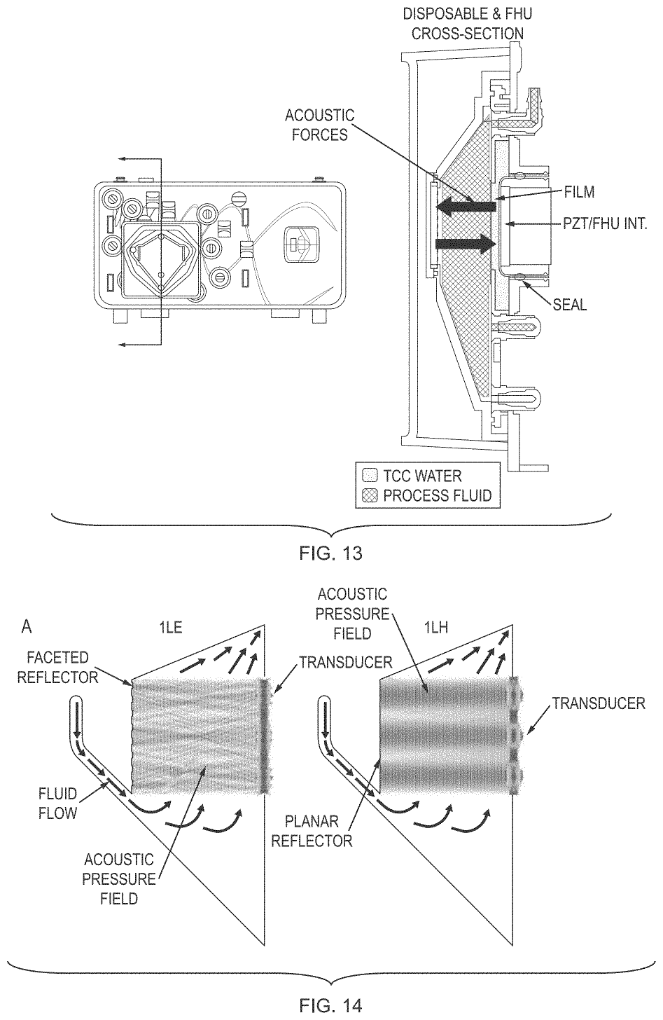

FIG. 13 is a front elevation view of the FHU with a portion of the FPE superimposed on the front interface panel and a sectional view of the portion of the FPE. The sectional view of the portion of the FPE shows the fluid chamber for housing the fluid being processed, which is exposed to the acoustic field generated by the ultrasonic transducer and reflector on either side of the fluid chamber. Also shown is a portion of the cooling loop that passes across the face of the ultrasonic transducer. The cooling loop portion is implemented with a thin, acoustically transparent film that overlays and is spaced from the face of the ultrasonic transducer. The film provides a border for a cooling fluid chamber formed in conjunction with the face of the ultrasonic transducer. The cooling fluid chamber permits cooling fluid to be flowed across the face of the ultrasonic transducer to remove thermal energy. The cooling fluid chamber is acoustically transparent to permit the ultrasonic transducer to generate an acoustic wave therethrough. The cooling fluid chamber is connected to the cooling fluid inlet and outlet on the front panel interface to permit the formation of a cooling loop with the FHU. The FPE includes seals that compress around the ultrasonic transducer when the FPE is seated to the front panel interface to obtain a fluid-tight seal for the cooling fluid chamber around the ultrasonic transducer.

FIG. 14 illustrates an example implementation of the fluid chamber of the FPE with different reflectors. The fluid chamber on the left-hand side of FIG. 14 includes a faceted reflector that disperses or scatters an incident acoustic wave, resulting in the illustrated dispersed acoustic pressure field. The fluid chamber on the right-hand side of FIG. 14 includes a planar reflector that reflects an incident acoustic wave back in a directly opposite direction, resulting in the illustrated acoustic pressure field. The FPE can be configured with different types of reflectors to contribute to generating different types of acoustic pressure fields within the fluid chamber to contribute to manipulating particles in the fluid according to a desired application.

FIG. 15 illustrates a particle concentration operation being implemented with the fluid chambers of FIG. 14. FIG. 15 shows the provision of a feed material to the fluid chamber, where the particles are captured and concentrated in the acoustic field generated within the fluid chamber. These concentrated particles can be subjected to a wash or a media exchange operation by providing a media to a lower portion of the fluid chamber, such that the media passes through the concentrated particles and through the acoustic field so that media flows out of the top of the fluid chamber, while particles are retained in or below the acoustic field.

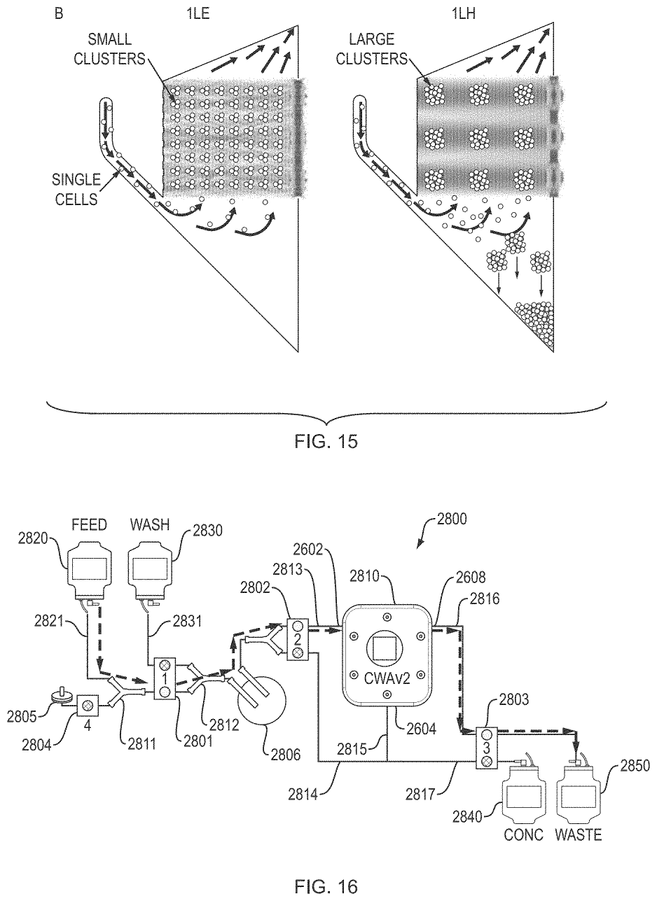

FIGS. 16-18 illustrate a concentrate-wash operation that can be implemented on the fluid processing workstation. The fluid connections, pathways and fluid chamber illustrated in FIGS. 16-18 are implemented within the FPE. The valves, pump and ultrasonic transducer illustrated in FIGS. 16-18 are implemented in the FHU, and interact with the tubing and fluid chamber of the FPE that is mounted to the front panel interface of the FHU.

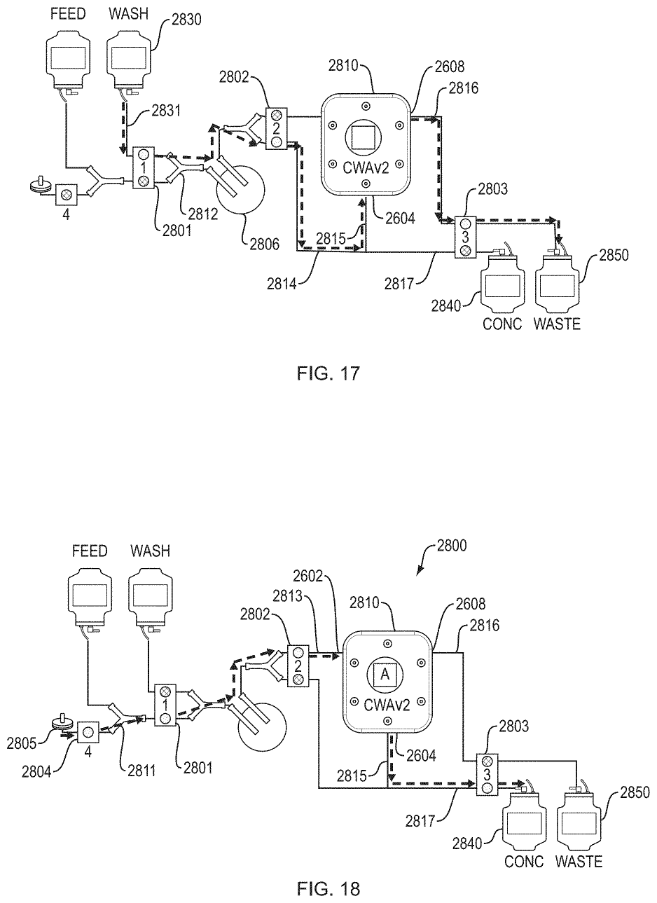

In FIG. 16, a particle-fluid mixture is provided to the fluid chamber in which an acoustic field is established via the ultrasonic transducer and reflector. The acoustic field retains and concentrates the particles in the mixture while passing the host fluid to an outlet that is directed to a waste container. The valve configuration and pump actuation are implemented by the FHU to create the fluid pathway through the FPE as shown in FIG. 16. The SCU provides control signals to the FHU to implement the valve and pump configuration to accomplish the wash operation, in accordance with a control configuration that can be selected by an operator. In FIG. 17, a wash media is supplied to a lower portion of the fluid chamber while the acoustic field is active. The wash media displaces the fluid in which the particles were entrained, urging the prior fluid to pass through the acoustic field that retains the particles, and out of an outlet at a top of the fluid chamber to a waste container. Again, the FHU causes actuation of the valves and pump under direction of the SCU to form the fluid pathway through the FPE for the wash operation. FIG. 18 illustrates a configuration for a collection of the concentrated and washed particles, where the fluid containing the concentrated and washed particles is drained from a bottom of the fluid chamber to a collection receptacle, such as a bag. The valves and pump are actuated by the FHU under control of the SCU.

The fluid pathways in the concentrate-wash operation illustrated in FIGS. 16-18 are implemented in a closed environment that is isolated from the valves and pumps, and completely contained within the FPE. Accordingly the configuration of the consumable FPE used in conjunction with the reusable FHU permits processing of particles in an isolated environment that can be sterile or otherwise disassociated from external exposure.

In an example implementation, the FPE may house the ultrasonic transducer and the FHU may house and the reflector, so that the ultrasonic transducer is disposable and the reflector is reusable. In such a configuration, the front panel interface may include an electrical connection interface that permits and RF power signal to be supplied from the FHU to the FPE to drive the ultrasonic transducer. Alternately, such a configuration may be implemented with a BNC connector on the FPE to accept a coaxial cable from the SCU or FHU that can provide the RF power signal to the ultrasonic transducer, as well as permit the SCU or FHU to receive feedback data from the ultrasonic transducer. Such a configuration would rely on the operator to connect the coaxial cable to the BNC connector on the installed FPE.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

1. A workstation for processing particles entrained in a fluid, comprising:
a consumable portion that includes a fluid chamber and a first acoustic component for contributing to generating an acoustic field in the fluid chamber;
a reusable portion that includes a second acoustic component for contributing to generating an acoustic field in the fluid chamber in conjunction with the first acoustic component; and
the consumable portion being removably mountable to the reusable portion.

2. The workstation of claim 1, wherein the first acoustic component comprises a reflector and the second acoustic component comprises an ultrasonic transducer.

3. The workstation of claim 1, wherein the reusable portion comprises a valve and the consumable portion includes a fluid pathway in fluid communication with the fluid chamber, the valve being configured to influence fluid flow in the fluid pathway when the consumable portion is mounted to the reusable portion.

4. The workstation of claim 1, further comprising registration structures on the consumable portion and the reusable portion that respectively cooperate to align the consumable portion when mounted to the reusable portion.

5. The workstation of claim 1, wherein the reusable portion comprises an interface for cooperatively engaging with the consumable portion, the interface including projections that project into openings in the consumable portion when the consumable portion is mounted to the reusable portion.

6. The workstation of claim 5, wherein at least one projection is a valve that engages with a fluid path in a corresponding opening of the consumable portion to permit the valve to influence fluid flow in the fluid path.

7. The workstation of claim 5 wherein at least one projection is an ultrasonic transducer that engages with the fluid chamber.

8. The workstation of claim 5 wherein at least one projection is a sensor that engages with a fluid path in a corresponding opening of the consumable portion to permit the sensor to sense a condition of the fluid path.

9. A method for processing a particle-fluid mixture, comprising:
removably mounting a consumable portion of a workstation that includes a fluid chamber and a first acoustic component for contributing to generating an acoustic field in the fluid chamber to a reusable portion of the workstation that includes a second acoustic component for contributing to generating an acoustic field in the fluid chamber in conjunction with the first acoustic component; and
flowing the mixture through the consumable portion under control of the reusable portion to implement a predefined process on the mixture.

10. The method of claim 9, wherein the first acoustic component comprises a reflector and the second acoustic component comprises an ultrasonic transducer.

11. The method of claim 9, wherein the reusable portion comprises a valve and the consumable portion includes a fluid pathway in fluid communication with the fluid chamber, the valve being configured to influence fluid flow in the fluid pathway when the consumable portion is mounted to the reusable portion.

12. The method of claim 9, further comprising removably mounting the consumable portion to the reusable portion in accordance with registration structures on the consumable portion and the reusable portion to align the consumable portion with the reusable portion.

13. The method of claim 9, further comprising removably mounting the consumable portion to an interface of the reusable portion to obtain a cooperative engagement such that projections in the interface project into openings in the consumable portion.

14. The method of claim 13, wherein at least one projection is a valve that engages with a fluid path in a corresponding opening of the consumable portion and actuating the valve to influence fluid flow in the fluid path.

15. The method of claim 13, wherein at least one projection is an ultrasonic transducer that engages with the fluid chamber.

16. The method of claim 13 wherein at least one projection is a sensor that engages with a fluid path in a corresponding opening of the consumable portion and actuating the sensor to sense a condition of the fluid path.

17. The method of claim 9, wherein the particles in the mixture comprise one or more of Chinese hamster ovary (CHO) cells, NS0 hybridoma cells, baby hamster kidney (BHK) cells, human cells, T cells, B cells, NK cells, algae, bacteria, viruses, apheresis material, iPSCs, hPSCs, or other cells grown as aggregates, or microcarriers.

18. A workstation for implementing a cell concentrate-wash process, comprising:
a system control unit that includes an operator interface and an acoustic driver module;
a fluid handling unit that includes an ultrasonic transducer in an interface, the ultrasonic transducer being electrically coupled to the acoustic driver module; and
a consumable cartridge that includes a fluid chamber and that is removably mountable to the interface such that the ultrasonic transducer can generate an acoustic wave in the fluid chamber.

19. The workstation of claim 18, wherein the consumable cartridge comprises a reflector that is opposed to the ultrasonic transducer when the consumable cartridge is removably mounted to the interface.

20. The workstation of claim 18, further comprising a cooling loop for removing thermal energy from the ultrasonic transducer, with a pathway that extends from the interface, through the consumable cartridge and back to the interface.
